(19) United States
(12) Patent Application Publication
(10) Pub. No.: US 2008/0249072 A1
(43) Pub. Date: Oct. 9, 2008

(54) Use of Low Dose Chemically Modified Tetracylines to Reduce Inflammatory Mediators

(76) Inventors: Lorne M. Golub, Smithtown, NY (US); Maria Emanuel Ryan, Laurel Hollow, NY (US); Hsi Ming Lee, Setauket, NY (US)

Correspondence Address: Ronald J Baron;Hoffmann & Baron, 6900 Jericho Turnpike, Syosset, NY, 11791, US

(21) Appl. No.: 11/663,325

(22) PCT Filed: Sep. 21, 2005
(86) PCT No.: PCT/US2005/033630

§ 371 Date: Jan. 30, 2008

(51) Int. Cl. A61K 31/65; A61P 1/02; A61P 9/12; A61P 29/00

(52) U.S. Cl. 514/152

(57) ABSTRACT

The invention is a method for reducing the production of inflammatory mediators, and/or treating conditions characterized by increased levels of inflammatory mediators, in a mammal in need thereof, by administering a 4-dedimethlaminosancycline to the mammal.

### BACKGROUND OF THE INVENTION

This invention relates to methods for reducing the production of biological molecules in response to inflammation.

Inflammation, in general, is the response of living tissue to damage. The damage that causes inflammation may be due to, for example, physical trauma, chemical substances, micro-organisms or other agents.

Chronic inflammation is a characteristic of many conditions including, for example, cardiovascular and cerebrovascular diseases (e.g. acute coronary syndromes and stroke), periodontitis, arthritis and inflammatory bowel syndrome. In response to inflammation, biological molecules are released by cells. One class of such molecules is inflammatory mediators.

Inflammatory mediators (e.g. cytokines) represent a broad family of proteins which regulate inflammatory and immune responses. Expression of inflammatory mediators has been found to be a universal response to different types of systemic and immune challenges, including the conditions mentioned above.

The class of inflammatory mediators includes, for example, Interleukin-1 (IL 1), Interleukin-6 (IL-6), tumor necrosis factor-alpha (TNF-α), and macrophage chemotactic protein-1 (MCP-1).

Prior art treatments for reducing the production of inflammatory mediators are limited and not without adverse effects. Hence, there is a need for a novel, alternate, and superior treatment for reducing the production of inflammatory mediators.

The compound tetracycline is a member of a class of antibiotic compounds that is referred to as the tetracyclines, tetracycline compounds, tetracycline derivatives and the like. The compound tetracycline exhibits the following general structure:

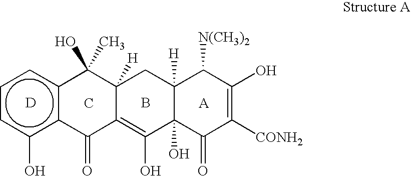

The numbering system of the tetracycline ring nucleus is as follows:

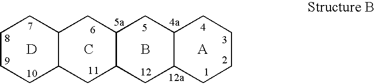

Tetracycline, as well as its terramycin and aureomycin derivatives, exist in nature, and are well known antibiotics. Natural tetracyclines may be modified without losing their antibiotic properties, although certain elements must be retained. The modifications that may and may not be made to the basic tetracycline structure have been reviewed by Mitscher in The Chemistry of Tetracyclines, Chapter 6, Marcel Dekker, Publishers, New York (1978). According to Mitscher, the substituents at positions 5-9 of the tetracycline ring system may be modified without the complete loss of antibiotic properties.

Changes to the basic ring system or replacement of the substituents at one or more of positions 4 and 10-12a, however, generally lead to synthetic tetracyclines with substantially less or effectively no antimicrobial activity. Some examples of chemically modified non-antibacterial tetracyclines (hereinafter CMTs) are 4-dedimethylaminotetracyline, 4-dedimethylaminosancycline(6-demethyl-6-deoxy-4-dedimethylaminotetracycline), 4-dedimethylaminominocycline(7-dimethylamino-6-demethyl-6-deoxy-4-dedimethylaminotetracycline), and 4-dedimethylaminodoxycycline(5-hydroxy-6-deoxy-4-dedimethylaminotetracycline).

In addition to their antimicrobial properties, tetracyclines have been described as having a number of other uses. For example, tetracyclines are also known to inhibit tumor necrosis factor (TNF), and interleukin-1 (IL-1). These properties cause the tetracyclines to be useful in treating a number of diseases.

As with any therapeutic compound, there are inherent advantages of using lower doses. Some of these advantages include lower cost, fewer side effects (to the extent there are any), etc.

The object of this invention is to provide new methods for reducing the production of inflammatory mediators.

### SUMMARY OF THE INVENTION

It has now been discovered that these and other objectives can be achieved by the present invention. In one embodiment, the invention provides a method for reducing the production of an inflammatory mediator selected from IL-1, IL-6, TNF-α and MCP-1 in a mammal in need thereof. In another embodiment, the invention provides methods for treating conditions characterized by increased levels of inflammatory mediators.

The methods comprise administering to the mammal an amount of a 4-dedimethlaminosancycline sufficient to produce a serum level thereof of approximately 0.1 to 1.1 μg/ml.

### BRIEF DESCRIPTION OF THE FIGURES

FIG. 1 compares the effectiveness of Doxycycline and CMT-3 at various dosages in inhibiting the production of Interleukin-6 (IL-6) in cells stimulated by c-reactive protein.

FIG. 2 compares the effectiveness of Doxycycline and CMT-3 at various dosages in inhibiting the production of Tumor Necrosis Factor-α (TNF-α) in cells stimulated by c-reactive protein.

### DETAILED DESCRIPTION OF THE INVENTION

It has now been discovered that the production of inflammatory mediators in a mammal in need thereof can be reduced by administering an unexpectedly low dose of a 4-dedimethylaminosancycline to the mammal. Inflammatory mediators are produced in the body by cells. Under normal conditions, inflammatory mediators are present in the body in very low concentrations. The cells that produce inflammatory mediators include, for example, monocytes and macrophages.

As used herein, the term “inflammatory mediator” refers to IL-1, IL-6, TNF-α, and MCP-1. IL-1, IL-6, TNF-α, and MCP-1 are believed to mediate tissue damage during inflammatory diseases.

The invention also relates to methods for treating mammals suffering from conditions characterized by increased levels of IL-1, IL-6, TNF-α, or MCP-1. A mammal in need of such treatments is any mammal that has an elevated level of one or more inflammatory mediators. In this specification, an elevated level of an inflammatory mediator is a level of the inflammatory mediator that is higher than normal, including a level that is pathologically higher than normal.

Mammals that can benefit from the methods of the invention include, for example, humans, farm animals, domestic animals, laboratory animals, etc. Some examples of farm animals include cows, pigs, horses, goats, etc. Some examples of domestic animals include dogs, cats, etc. Some examples of laboratory animals include rats, mice, rabbits, guinea pigs, etc.

The inflammation can be acute or chronic. Acute inflammation is short-lasting, lasting only a few days or weeks. An acute inflammatory condition is any condition that involves a short term production of inflammatory mediators, e.g. wounds and acute abscess.

If a condition is longer lasting however, then it is referred to as chronic inflammation. A chronic inflammatory condition is any condition that involves the chronic or long lasting production of inflammatory mediators. Chronic inflammatory conditions can last weeks, months or years. Some examples of chronic inflammatory conditions include arthritis, cardiovascular and cerebrovascular disease, periodontitis, inflammatory bowel disease, non-healing wounds and non-healing ulcers.

The methods of the invention comprise administration of a 4-dedimethlyaminosancycline. The 4-dedimethlyaminosancyclines are a family of chemically modified tetracyclines (CMTs) having Structure A below, wherein R⁷, R⁸ and R⁹ may be un substituted (i.e. R⁷, R⁸, and R⁹ all represent hydrogen), or one, two or three of R⁷, R⁸ and R⁹ are independently substituted by, for example, hydroxyl, C₁-C₄ straight chain or branched lower alkyl, amino, methylamino, dimethylamino, azido, acylamino, nitro, (N,N-dimethyk)glycylamino, ethoxythiocarbonylthio, diazonium, halo (i.e. fluoro, chloro, bromo, or iodo), acetamido, dimethylaminoacetamido, palmitamido, CONHCH₂-pyrrolidin-1-yl, CONHCH₂- piperadin-1-yl, CONHCH₂-morpholin-1-yl, or CONHCH₂-piperazin-1-yl.

Structure A represents the parent 4-dedimethylaminosancycline compound.

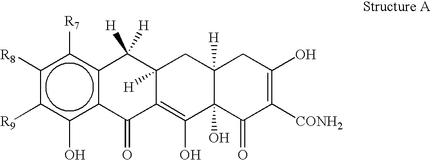

In a preferred embodiment of Structure A, R⁷, R⁸ and R⁹ taken together in each case have the following meanings:

| | R⁷ | | R⁸ | R⁹ |
|---|---|---|---|---|
| | azido | | hydrogen | hydrogen |
| | dimethylamino | | hydrogen | azido |
| | hydrogen | | hydrogen | azido |
| | dimethylamino | | hydrogen | amino |
| | acylamino | | hydrogen | hydrogen |
| | amino | | hydrogen | nitro |
| | hydrogen | | hydrogen | (N,Ndimethyl)glycylamino |
| | amino | | hydrogen | amino |
| | hydrogen | | hydrogen | ethoxythiocarbonylthio |
| | dimethylamino | | hydrogen | acylamino |
| | dimethylamino | | hydrogen | diazonium |
| | dimethylamino | | chloro | amino |
| | hydrogen | | chloro | amino |
| | amino | | chloro | amino |
| | acylamino | | chloro | acylamino |
| | amino | | chloro | hydrogen |
| | acylamino | | chloro | hydrogen |
| | monoalkylamino | | chloro | amino |
| | nitro | | chloro | amino |
| | dimethylamino | | chloro | acylamino |
| | dimethylamino | | chloro | dimethylamino |
| | acylamino | | hydrogen | hydrogen |
| | hydrogen | | hydrogen | acylamino |
| (CMT-3) | hydrogen | | hydrogen | hydrogen |
| (CMT-301) | bromo | | hydrogen | hydrogen |
| (CMT-302) | nitro | | hydrogen | hydrogen |
| (CMT-303) | hydrogen | | hydrogen | nitro |
| (CMT-304) | acetamido | | hydrogen | hydrogen |
| (CMT-305) | hydrogen | | hydrogen | acetamido |
| (CMT-306) | hydrogen | | hydrogen | dimethylamino |
| (CMT-307) | amino | | hydrogen | hydrogen |
| (CMT-308) | hydrogen | | hydrogen | amino |
| (CMT-309) | hydrogen | | hydrogen | dimethylaminoacetamido |
| (CMT-310) | dimethylamino | | hydrogen | hydrogen |
| (CMT-311) | hydrogen | | hydrogen | palmitamide |
| (CMT-312) | hydrogen | hydrogen | hydrogen | CONHCH₂-pyrrolidin-1-yl |
| (CMT-313) | hydrogen | hydrogen | hydrogen | CONHCH₂-piperadin-1-yl |
| (CMT-314) | hydrogen | hydrogen | hydrogen | CONHCH₂-morpholin-1-yl |
| (CMT-315) | hydrogen | hydrogen | hydrogen | CONHCH₂-piperazin-1-yl |

Chemically modified tetracyclines, such as 4-dedimethylaminosancycline, can be made by methods known in the art. See, for example, Mitscher, L.A., The Chemistry of the Tetracycline Antibiotics, Marcel Dekker, New York (1978), Ch. 6, and U.S. Pat. Nos. 4,704,383, 5,532,227, and 6,506,740.

The methods of the invention also include the administration of pharmaceutically acceptable salts of 4-dedimethylaminosancycline, including acid-addition and metal salts. Such salts are formed by well known procedures. By “pharmaceutically acceptable salts” is meant salts that do not substantially contribute to the toxicity of the compound.

Some examples of suitable salts include salts that can be formed by mixing solutions of basic tetracycline compounds and mineral acids. Some examples of suitable mineral acids include hydrochloric, hydroiodic, hydrobromic, phosphoric, metaphosphoric, nitric and sulfuric acids, as well as salts of organic acids such as tartaric, acetic, citric, malic, benzoic, glycollic, gluconic, gulonic, succinic, and the like.

After preparation, a 4-dedimethylaminosancycline of the present invention can be conveniently purified by standard methods known in the art. Some suitable examples include crystallization from a suitable solvent or partition-column chromatography.

The preferred pharmaceutical composition for use in the methods of the invention comprises a 4-dedimethylaminosancycline in a suitable pharmaceutical carrier (vehicle) or excipient as understood by practitioners in the art. Examples of carriers and excipients include starch, milk, sugar, certain types of clay, gelatin, stearic acid or salts thereof, magnesium or calcium stearate, talc, vegetable fats or oils, gums and glycols.

The 4-dedimethylaminosancycline may be administered by methods known in the art, typically, systemically. Systemic administration can be enteral or parenteral. Oral administration is a preferred route of delivery of the 4-dedimethylaminosancycline. Pharmaceutical compositions comprising the 4-dedimethylaminosancycline compound, and appropriate diluents, carriers, and the like, are readily formulated. Liquid or solid formulations can be employed. Some suitable examples of solid formulations include tablets and capsules, such as gelatin capsules, etc.

Administration can also be accomplished by a nebulizer or liquid mist. Nebulization is a preferred route of delivery of the 4-dedimethylaminosancycline in situations where the respiratory system is particularly infected. By utilizing a nebulizer, the 4-dedimethylaminosancycline is taken directly into the individuals respiratory system through inspiration.

Parenteral administration of the 4-dedimethylaminosancycline (e.g., intravenous, intramuscular, subcutaneous injection) is also contemplated. Formulations using conventional diluents, carriers, etc. such as are known in the art can be employed to deliver the compound.

The 4-dedimethylaminosancycline may be administered to mammals by sustained release, as is known in the art. Sustained release administration is a method of drug delivery to achieve a certain level of the drug over a particular period of time. See, for example, U.S. Published Patent Application No. 10/474,240 filed Oct. 3, 2003.

The amount of 4-dedimethylaminosancycline administered is any amount sufficient to produce a serum level thereof of approximately 0.1 to 1.1 μg/ml. For purposes of this application, “serum level” means the concentration of the 4-dedimethylaminosancycline measured in a patient's blood sample taken twenty four (24) hours after the dose taken on day seven of a treatment regimen.

For example, on the eighth day (after a patient has taken a particular dose of the 4-dedimethylaminosancycline for the previous seven days), the minimum serum concentration of the 4-dedimethylaminosancycline is approximately 0.1, 0.2, 0.3, 0.4 or 0.5 μg/ml, and the maximum serum concentration of the 4-dedimethylamonisancycline is approximately 0.7, 0.8, 0.9, 1.0, or 1.1 μg/ml. The serum level can be measured periodically depending on the length of treatment. For example, treatment can last for weeks, or months or years.

The amount of 4-dedimethylaminosancycline administered that is sufficient to produce a serum concentration of between about 0.1 and 1.1 μg/ml is any daily dose that achieves the desired serum concentration. Such doses can be readily determined by those skilled in the art. The actual daily dose of the 4-dedimethylaminosancycline administered in a specified case will vary according to the particular compositions formulated, the mode of application (e.g. frequency and duration of administration), and the particular subject being treated (e.g. size and age).

The minimum amount of the 4-dedimethylaminosancycline administered to a human is the lowest amount capable of providing a sufficient serum level as described above. For example, a suitable minimum amount is 1, 2, 4, or 6 mg/day.

The maximum amount for a mammal is the highest amount that achieves a sufficient serum level as described above. For example, a suitable maximum amount is 20, 18, 12, or 10 mg/day.

Suitable ranges of serum levels and daily doses can be obtained by combining any of the minimum amounts described above with any of the maximum amounts described above. In general, the 4-dedimethylaminosancycline is administered in an amount which results in a serum concentration between about 0.1and 1.1 μg/ml, more preferably between about 0.3 and 0.8 μg/ml. For example, CMT-3, is preferably administered in an amount which results in a serum concentration between about 0.1 and 0.8 μg/ml, more preferably between about 0.4 and 0.7 μg/ml.

Ordinarily, one expects drugs to manifest their activity in a dose dependent manner. Thus, examples 1-3 below demonstrate that the inhibition by doxycycline of the production of IL-6, TNF-α and MCP-1 is dose response in the usual way. For example, as the concentration of doxycycline administered increases, the inhibition of inflammatory mediators increases.

It has been unexpectedly discovered, however, that unusually low doses (e.g. doses that result in serum levels of 0.1 to 1.1 μg/ml) of a 4-dedimethylaminosancycline are particularly effective in reducing the production of inflammatory mediators. As can be seen in the examples to follow, CMT-3 is more effective at a concentration of 0.5 μg/ml than at a concentration of 1.5 μg/ml in inhibiting IL-6, TNF-α and MCP-1.

Thus, the efficacy of CMT-3 surprisingly is not a simple dose-response. Although the highest concentration of CMT-3 (5.0 μg/ml) was the most effective in reducing the inflammatory mediators, the lowest concentration of CMT-3 tested (0.5 μg/ml) was actually MORE effective than a 3-fold higher (1.5 μg/ml) concentration of CMT-3.

### EXAMPLES

Experiments were conducted to compare the efficacy of two tetracycline compounds, doxycycline and CMT-3, on reducing the production of inflammatory mediators, TNF-α, IL-6 and MCP-1 produced by monocytes/macrophages. Doxycycline is a well known tetracycline antibiotic. CMT-3 is a non-antibacterial 4-dedimethylamino derivative of sancycline. See above.

The experiments utilized a cell culture stimulated by biologic factors that cause inflammation, such as the inflammation seen in, for example, coronary and cerebro-vascular disease. Units of human blood were obtained and peripheral blood monocytes were separated by Ficoll-Hypaque gradient centrifugation. Viable human monocytes/macrophages were incubated in cell culture under standard sterile conditions on plastic in RPMI media at 37° C. for 18 hours.

The monocytes/macrophages were incubated under the following experimental conditions:
(i) cells alone;
(ii) cells stimulated by c-reactive protein (CRP)/Oxidized LDL cholesterol complex (CRP/Oxid. LDL complex), no tetracycline present;
(iii) cells stimulated by CRP/Oxid. LDL complex with doxycycline at concentrations of 0.7 μg/ml, 5.0 μg/ml and 10.0 μg/ml; and.
(iv) cells stimulated by CRP/Oxid. LDL complex with CMT-3 at concentrations of 0.5 μg/ml, 1.5 μg/ml and 5.0 μg/ml.

CRP/Oxid. LDL complex stimulates monocytes/macrophages to secrete excessive levels of cytokines (e.g. IL-1, IL-6, TNF-α, and MCP-1). Excessive levels of cytokines result in the rupture of atheroscleromatus plaques lining coronary and cerebral arteries causing thrombosis, heart attack and stroke.

At the end of an 18 hour incubation, the conditioned media were collected and analyzed by ELISA for inflammatory mediators IL-6, TNF-α and MCP-1. The results are described in examples 1-3.

### Example 1

### Interleukin-6 (IL-6)

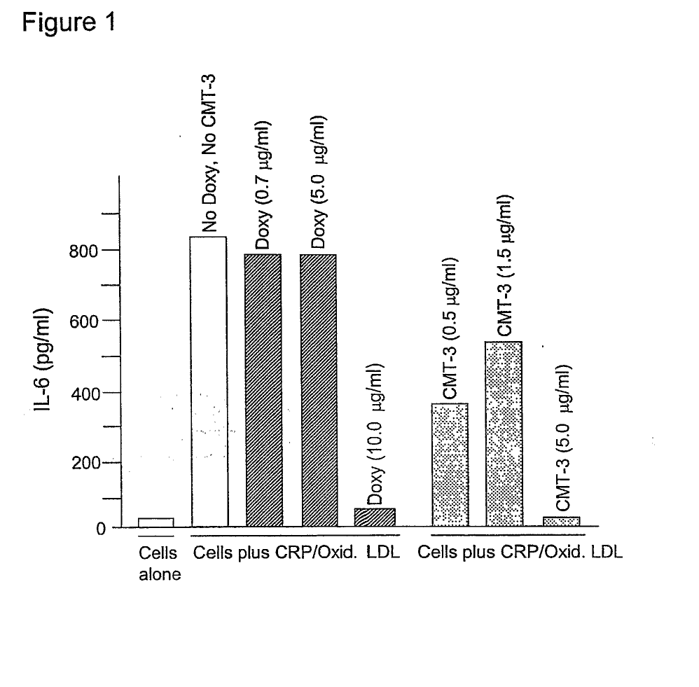

The monocytes/macrophages alone synthesized and secreted small but detectable amounts of IL-6 (approx. 15 pg/ml) into the extracellular media. However, those cells that were cultured with the CRP/Oxid. LDL complex showed a dramatic (5.233%) increase in the production of IL-6. (See FIG. 1).

As expected, doxycycline inhibited the production of IL-6 in a dose-response fashion. At 0.7 μg/ml, little or no effect was seen on IL-6 production. (FIG. 1). At 5.0 μg/ml, no further effect was seen on IL-6. At a concentration of 10.0 μg/ml, which is a very high (possibly toxic) concentration of doxycycline in the blood stream, the production of IL-6 was reduced to essentially normal levels. (FIG. 1).

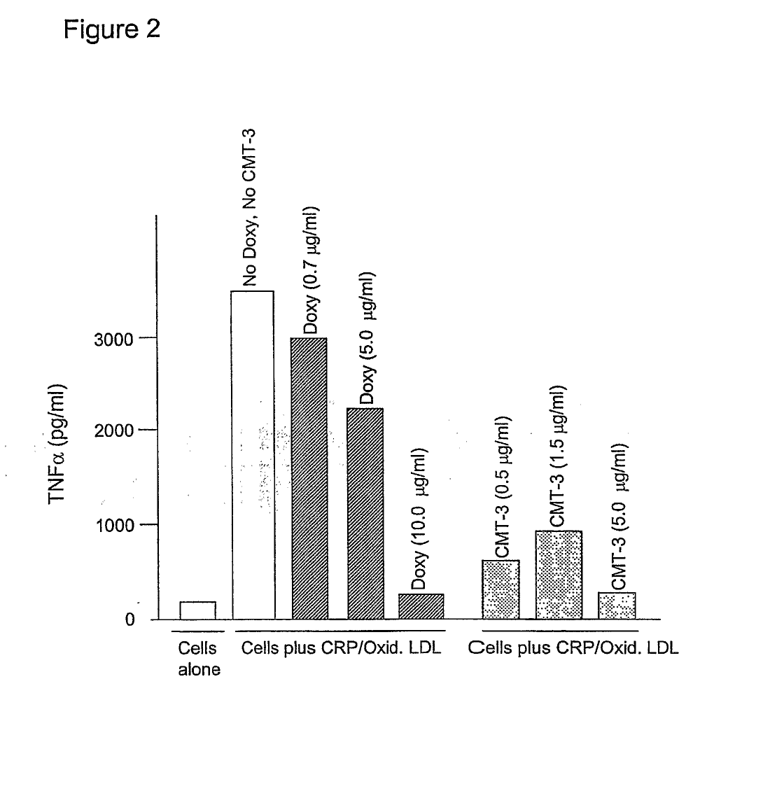

CMT-3 was more potent than doxycycline because it reduced IL-6 production at all three concentrations tested (0.5, 1.5 and 5.0 μg/ml). See FIG. 2. IL-6 production, in the presence of 1.5 μg/ml CMT-3, was 43% higher than the production of IL-6 in the presence of the much lower concentration (0.5 μg/ml) of CMT-3.

### Example 2

### Tumor Necrosis Factor-α (TNF-α)

The monocytes/macrophages alone synthesized and secreted small but detectable amounts of TNF-α (approx. 200 pg/ml) into the extracellular media. However, those cells that were cultured with the CRP/Oxid. LDL complex showed a dramatic (1,650%) increase in the production of TNF-α. (See FIG. 2).

As expected, doxycycline inhibited the production of TNF-α in a dose-response fashion. At 0.7 μg/ml, TNF-α production was inhibited by about 14%. (FIG. 2). At 5.0 μg/ml, TNF-α production was inhibited by about 37%. At a concentration of 10.0 μg/ml, which is a very high (possibly toxic) concentration of doxycycline in the blood stream, the production of TNF-α was reduced by about 94%, to essentially normal levels. (FIG. 2).

CMT-3 was more potent than doxycycline because it reduced TNF-α production at all three concentrations tested (0.5, 1.5 and 5.0 μg/ml). See FIG. 2. TNF-α production, in the presence of 1.5 μg/ml CMT-3, was about 50% higher than the production of this cytokine in the presence of the much lower concentration (0.5 μg/ml) of CMT-3.

### Example 3

### Macrophage Chemotactic Protein-1 (MCP-1)

The monocytes/macrophages alone synthesized and secreted small but detectable amounts of MCP-1. However, those cells that were cultured with the CRP/Oxid. LDL complex showed a dramatic (500%) increase in the production of MCP-1.

Doxycycline inhibited the production of MCP-1 in a dose-response fashion. At 0.5 μg/ml and at 1.5 μg/ml, doxycycline did not inhibit MCP-1 production. At 5.0 μg/ml, TNF-α production was inhibited by about 68%.

MCP-1 production, in the presence of 1.5 μg/ml CMT-3, was 16% higher than the production of MCP-1 in the presence of the much lower concentration (0.5 μg/ml) of CMT-3.

We claim:

1. A method for reducing production of an inflammatory mediator selected from IL-1, IL-6, MCP-1 and TNF-α in a mammal in need thereof, wherein the method comprises administering to the mammal an amount of a 4-dedimethylaminosancycline sufficient to produce a serum level thereof of approximately 0.1 to 1.1 μg/ml.

2. The method according to claim 1, wherein the 4-dedimethylaminosancycline is CMT-3.

3. The method according to claim 1, wherein the amount of the 4-dedimethylaminosancycline is approximately 1.0 to 20 mg/day.

4. The method according to claim 1, wherein the inflammatory mediator is IL-1.

5. The method according to claim 1, wherein the inflammatory mediator is IL-6.

6. The method according to claim 1, wherein the inflammatory mediator is TNF- α.

7. The method according to claim 1, wherein the inflammatory mediator is MCP-1.

8. The method according to claim 1, wherein the production of the inflammatory mediator occurs in monocytes.

9. A method for treating a condition characterized by increased IL-1 levels in a mammal in need thereof, wherein the method comprises administering to the mammal an amount of a 4-dedimethylaminosancycline sufficient to produce a serum level thereof of approximately 0.1 to 1.1 μg/ml.

10. The method according to claim 9, wherein the amount of the 4-dedimethylaminosancycline is approximately 1.0 to 20 mg/day.

11. The method according to claim 9, wherein the 4-dedimethylaminosancycline is CMT-3.

12. The method according to claim 9, wherein the condition is a chronic inflammatory condition.

13. The method according to claim 9, wherein the condition is arthritis.

14. The method according to claim 9, wherein the condition is periodontitis.

15. The method according to claim 9, wherein the condition is cardiovascular and/or cerebrovascular disease.

16. The method according to claim 9, wherein the condition is inflammatory bowel disease.

17. A method for treating a condition characterized by increased IL-6 levels in a mammal in need thereof, wherein the method comprises administering to the mammal an amount of a 4-dedimethylaminosancycline sufficient to produce a serum level thereof of approximately 0.1 to 1.1 μg/ml.

18. The method according to claim 17, wherein the amount of the 4-dedimethylaminosancycline is approximately 1.0 to 20 mg/day.

19. The method according to claim 17, wherein the 4-dedimethylaminosancycline is CMT-3.

20. The method according to claim 17, wherein the condition is a chronic inflammatory condition.

21. The method according to claim 17, wherein the condition is arthritis.

22. The method according to claim 17, wherein the condition is periodontitis.

23. The method according to claim 17, wherein the condition is cardiovascular and/or cerebrovascular disease.

24. The method according to claim 17, wherein the condition is inflammatory bowel disease.

25. A method for treating a condition characterized by increased TNF-α levels in a mammal, wherein the method comprises administering to the mammal an amount of a 4-dedimethylaminosancycline sufficient to produce a serum level thereof of approximately 0.1 to 1.1 μg/ml.

26. The method according to claim 25, wherein the amount of the 4-dedimethylaminosancycline is approximately 1.0 to 20 mg/day.

27. The method according to claim 25, wherein the 4-dedimethylaminosancycline is CMT-3.

28. The method according to claim 25, wherein the condition is a chronic inflammatory condition.

29. The method according to claim 25, wherein the condition is arthritis.

30. The method according to claim 25, wherein the condition is periodontitis.

31. The method according to claim 25, wherein the condition is cardiovascular and/or cerebrovascular disease.

32. The method according to claim 25, wherein the condition is inflammatory bowel disease.

33. A method for treating a condition characterized by increased MCP-1 levels in a mammal, wherein the method comprises administering to the mammal an amount of a 4-dedimethylaminosancycline sufficient to produce a serum level thereof of approximately 0.1 to 1.1 μg/ml.

34. The method according to claim 33, wherein the amount of the 4-dedimethylaminosancycline is approximately 1.0 to 20 mg/day.

35. The method according to claim 33, wherein the 4-dedimethylaminosancycline is CMT-3.

36. The method according to claim 33, wherein the condition is a chronic inflammatory condition.

37. The method according to claim 33, wherein the condition is inflammatory bowel disease.

38. The method according to claim 33, wherein the condition is arthritis.

39. The method according to claim 33, wherein the condition is periodontitis.

40. The method according to claim 33, wherein the condition is cardiovascular and/or cerebrovascular disease.
